(19) United States
(12) Patent Application Publication
(10) Pub. No.: US 2005/0234354 A1
(43) Pub. Date: Oct. 20, 2005

(54) System and method for assessing a patient's risk of sudden cardiac death

(76) Inventors: G. Ian Rowlandson, Milwaukee, WI (US); David E. Albert, Oklahoma City, OK (US)

Correspondence Address: MICHAEL BEST & FRIEDRICH LLP, 100 EAST WISCONSIN AVENUE, MILWAUKEE, WI, 53202-4108, US

(21) Appl. No.: 10/824,983

(22) Filed: Apr. 15, 2004

(51) Int. Cl. A61B 5/04

(52) U.S. Cl. 600/509

(57) ABSTRACT

A system and method for assessing a patient's risk of sudden cardiac death. One method of the invention can include acquiring patient data at one of a plurality of healthcare locations, identifying the patient as being worthy of an on-going sudden cardiac death risk assessment based on the acquired patient data, and performing the on-going sudden cardiac death risk assessment whenever new patient data is acquired at any one of the plurality of healthcare locations.

### BACKGROUND OF THE INVENTION

Sudden cardiac death (“SCD”) can be generally defined as death within one hour of the onset of symptoms, or death without symptoms, without a previously-known disease or a disease that was expected to be lethal. SCD is often described with respect to an unwitnessed death with the victim having been known to be alive less than 24 hours earlier.

SCD can kill its victims within minutes and often occurs in outwardly healthy people who have no known heart disease. Although it may occur in outwardly healthy people, most victims do have heart disease or other health problems, often without being aware of it. SCD claims about 300,000 lives a year in the United States and presents a public health challenge in that often the only indication a patient is at risk appears when the patient succumbs, without warning, to a heart failure episode.

In many cases, SCD victims suffer from ventricular tachycardia that degenerates into ventricular fibrillation. Ventricular tachycardia is a type of cardiac arrhythmia that is a serious, often-times, fatal condition characterized by rapid, uncontrolled, and ineffective beating of the heart. Ventricular fibrillation is a chaotic ventricular heart rhythm which produces little or no net blood flow from the heart, such that there is little or no net blood flow to the brain and other organs. Ventricular fibrillation, if not terminated, results in death. Researchers continue their efforts to predict the onset and triggers for such ventricular tachyarrhythmias and SCD.

The probability of SCD for a particular patient is not necessarily a static phenomenon. It can be dynamic and based on changes in the patient's status. Also, there can be multiple physiological reasons or root causes for SCD, and each can have a different measurement or indicator.

Conventionally, an assessment of the probability of SCD is not routinely made even after a significant change in patient status. Furthermore, it is often not known when the last time such an assessment has been done on a particular patient.

An assessment of SCD can be based in part on routine cardiac measurements. However, a caregiver may acquire routine cardiac data for other purposes and not be aware of the relevance of this data to the probability of SCD.

### BRIEF DESCRIPTION OF THE INVENTION

Some embodiments of the invention provide a method of assessing a risk of sudden cardiac death for a patient. The method can include acquiring patient data at one of a plurality of healthcare locations, identifying the patient as being worthy of an on-going sudden cardiac death risk assessment based on the acquired patient data, and performing the on-going sudden cardiac death risk assessment whenever new patient data is acquired at any one of the plurality of healthcare locations.

In some embodiments, the invention can include a method of identifying a patient for an on-going sudden cardiac death risk assessment. The method can include searching patient data stored in a hospital information system for a characteristic that identifies the patient as being worthy of an on-going sudden cardiac death risk assessment, and flagging an identification associated with the patient if the characteristic indicates that the patient is at risk for sudden cardiac death.

Other features and advantages of the invention will become apparent to those skilled in the art upon review of the following detailed description, claims and drawings.

### BRIEF DESCRIPTION OF THE DRAWINGS

FIG. 1 is a schematic illustration of a SCD assessment system according to one embodiment of the invention.

FIG. 2 is a flow chart of a SCD assessment method according to one embodiment of the invention.

### DETAILED DESCRIPTION OF THE INVENTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limited. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and can include electrical connections or couplings, whether direct or indirect.

In addition, it should be understood that embodiments of the invention include both hardware and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the invention may be implemented in software. As such, it should be noted that a plurality of hardware and software based devices, as well as a plurality of different structural components may be utilized to implement the invention. Furthermore, and as described in subsequent paragraphs, the specific mechanical configurations illustrated in the drawings are intended to exemplify embodiments of the invention and that other alternative mechanical configurations are possible.

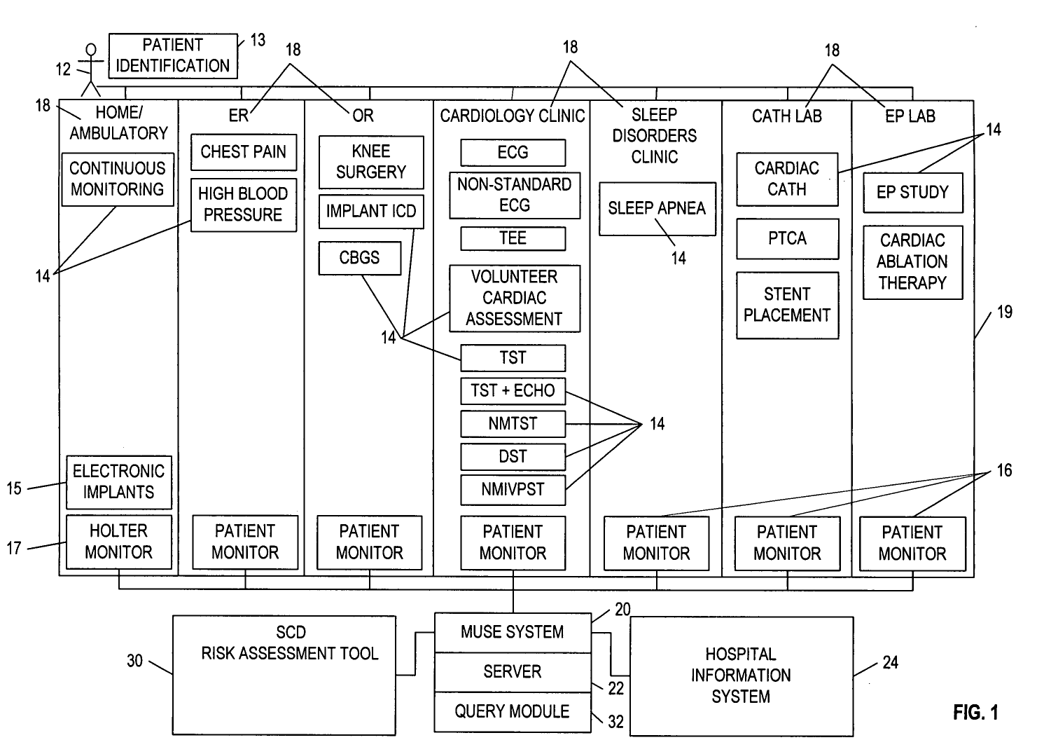

FIG. 1 illustrates a SCD assessment system 10 according to one embodiment of the invention. The SCD risk assessment system 10 can be associated with or connected to a patient 12 having a patient identification 13. The patient 12 may have had or may have in the future various procedures 14 performed to determine his or her cardiac health. The SCD risk assessment system 10 can include one or more patient monitors 16 at one or more locations 18 within or associated with a healthcare-providing system 19. The one or more patient monitors 16 can include at least one of a diagnostic tool, a data acquisition device, an imaging tool, a display, and a combination thereof. The patient 12 may have visited or may visit any one or more of the locations 18 regarding various symptoms, tests, and/or the procedures 14. The patient's symptoms, tests, and/or the procedures 14 can be recorded, performed, or administered at any of the locations 18 within the healthcare-providing system 19 and at various points in time (e.g., simultaneously or non-simultaneously). The patient monitors 16 can be associated with each location 18 for acquiring and/or displaying patient data. More than one patient monitor 16 per location 18 can be used. However, FIG. 1 illustrates only one patient monitor 16 for each location 18.

As also shown in FIG. 1, the SCD assessment system 10 can include a medical data information system, such as a MUSE system 20 (i.e., the MUSE® medical information system sold by GE Medical Systems Information Technologies, Inc.). The SCD assessment system 10 can further include a variety of other medical information systems, including without limitation, at least one of a Centricity DMS Server (i.e., the Centricity DMS® Server sold by GE Medical Systems Information Technologies, Inc.), and a Centricity IMS Server (i.e., the Centricity DMS® Server sold by GE Medical Systems Information Technologies, Inc.). In some embodiments, the SCD assessment system 10 can include an additional server that provides a single point of access to all of the medical information systems. For simplicity, the MUSE system 20 will be described herein, but one of ordinary skill in the art will appreciate that a variety of other medical information systems, or combinations thereof, can be used to store, retrieve, and edit patient records without departing from the spirit and scope of the invention.

The MUSE system 20 can allow healthcare providers to comment on patient records; authorize studies; enter, edit and/or confirm patient cardiology reports and original test data; generate reports based on test results; conduct searches; and manage a patient's cardiology information using any laptop or desktop computer, whether the computer is within the healthcare-providing entity or physically separated from the healthcare-providing entity.

As further shown in FIG. 1, the MUSE system 20 can include or can communicate with a server 22 that communicates with a hospital information system (HIS) 24. The hospital information system 24 can be a central information repository for an entire healthcare-providing system (i.e., a Clinical Data Repository). For example, the hospital information system 24 can contain patient information that can be accessed by a diverse population of administrative and medical staff members within a healthcare-providing system. The hospital information system 24 can be accessed from a laptop, a desktop, or any other workstation associated with any department or area within the healthcare-providing system including, without limitation, a pharmacy, a billing department, an accounting department, a nurses' station, an operating room, an emergency room, an intensive care unit, a cath lab, an x-ray room, or any other department or sector within the healthcare-providing system 19.

The patient monitors 16 associated with the locations 18 can communicate with the MUSE system 20, as shown schematically in FIG. 1, in order to store new test data and retrieve any portion of a patient's history. The MUSE system 20 can also communicate with a SCD risk assessment tool 30, which can be used to calculate a probability of SCD p for the patient 12. The SCD risk assessment tool 30 can calculate the probability of SCD p based on the patient's most recent medical data added to the MUSE system 20 or any portion of the patient's history stored in or accessible by the MUSE system 20. The SCD risk assessment tool 30 can be a multi-parameter system that calculates the probability of SCD p based on a variety of cardiological parameters, general patient health parameters, or any other suitable patient data. For example, in some embodiments of the invention, the SCD risk assessment tool 30 can calculate the probability of SCD p based on various aspects and features of a patient electrocardiogram (ECG), such as T-wave alternans (TWA), QRS duration, an ECG serial comparison, various arrythmias (such as ventricular tachycardia), heartrate turbulence, signal-averaged ECGs, rhythm abnormalities, ST/T measurements, heart rate variability, or any other ECG measurements or analysis techniques. In other embodiments of the invention, the SCD risk assessment tool 30 can calculate the probability of SCD p based on aspects and features of an echocardiogram, any form of cardiac imaging, blood pressure measurements, a stress test, a stress-echo test, a stress-nuclear test, a cardiac catheterization or angiogram, an electrophysiology study, or a Holter study, etc. In still other embodiments of the invention, the SCD risk assessment tool 30 can calculate the probability of SCD p based on patient data that is manually entered by a healthcare provider or automatically entered into any portion of the system 10. For example, the patient data can include measurements (respiration, temperature, carbon dioxide, oxygen saturation, weight, blood pressure, etc.), medical personnel conclusions, images, charts, graphs, identified abnormalities, patient identifiers (age, gender, race, etc.), symptoms, dates, and identification of prescribing, attending, or reading physicians.

The SCD risk assessment system 10 can be employed once the patient 12 has been identified as worthy of on-going SCD risk assessment. The patient 12 can be identified as worthy of on-going SCD risk assessment manually or automatically. In some embodiments of the invention, the SCD risk assessment system 10 can include a query module 32 that automatically searches the patient data stored in the MUSE system 20 or the patient's medical history in the hospital information system 24 for specific features or combinations of features that will identify the patient 12 as worthy of on-going SCD risk assessment. The query module 32 illustrated in FIG. 1 is shown as being associated with or connected to the MUSE system 20, but it should be understood that the query module 32 can be associated with or connected to the hospital information system 24, or other data repository systems that communicate, directly or indirectly, with the MUSE system 20.

In some embodiments, the query module 32 can automatically identify the patient 12 as being worthy of on-going SCD risk assessment depending on a variety of patient characteristics, such as the patient's age, gender, race, family history, weight, blood pressure, a previous cardiac arrhythmia, a low ejection fraction, a pre-existing condition (such as diabetes, mitral valve prolapse, etc.), a previous clinical event (such as an embolism, a heart attack, a stroke, etc.), patient lifestyle (such as stress level, drinking habits, smoking habits, etc.), etc.

The query module 32 can be programmed to run a query or a set of queries at suitable times to determine if the patient 12 is worthy of on-going SCD risk assessment. For example, the query module 32 can run a query each time the patient 12 is examined by a physician; admitted to a hospital; treated in the emergency room; has an electrocardiogram taken; and/or has any change in family history, lifestyle or any other relevant patient characteristic.

Alternatively, in some embodiments of the invention, the patient 12 can be manually identified as worthy of on-going SCD risk assessment by a healthcare provider. For example, the healthcare provider can flag the patient 12 (and in some embodiments, the patient identification 13) for on-going SCD risk assessment so that, from that point forward, the SCD risk assessment tool 30 calculates the probability of SCD p for the patient 12 each time a new piece of data or information is acquired from the patient 12 (or associated with the patient identification 13).

Once the patient 12 has been identified (i.e., automatically or manually) as being worthy of on-going SCD risk assessment, the patient 12, by way of the patient identification 13, can be flagged within the MUSE system 20, the hospital information system 24, or any other patient data repository that communicates, directly or indirectly, with the MUSE system 20. Once the patient 12 or the patient identification 13 has been flagged, the SCD risk assessment tool 30 can calculate the probability of SCD p for the patient 12 from that point forward each time any data is added to the MUSE system 20 for the patient 12 (e.g., every time any new data is acquired from the patient 12).

As shown in FIG. 1, the locations 18 within the healthcare-providing system 19 that the patient 12 visits can include, by way of example only, the patient's home (i.e., for on-going data recording over a period of time), an emergency room (ER), an operating room (OR), a cardiology clinic or department, a sleep disorders clinic, a catheterization lab, or an electrophysiology lab. The locations 18 can also include many other locations within the healthcare-providing system 19, or locations outside of the healthcare-providing system. For example, retired patients may visit a healthcare system in one state during the winter and a healthcare system in another state during the summer.

The patient 12 may have undergone continuous monitoring at home (or in any ambulatory situation) using an electronic implant 15 (e.g., a pacemaker, an implantable cardioverter defrillator (ICD), etc.) or a Holter monitor 17, as shown in FIG. 1. The Holter monitor 17 is a type of patient monitor 16 that allows a patient's ECG to be recorded for an extended period of time (e.g., 24 hours) and throughout normal daily activities, such as eating, sleeping and exercising.

The patient 12 may also have visited the emergency room for chest pain or symptoms of high blood pressure. If the patient 12 was not already identified as worthy of on-going SCD risk assessment (and the patient identification 13 flagged accordingly in the SCD risk assessment system 10), a healthcare provider in the emergency room can manually identify the patient 12 as being worthy of the on-going assessment. Alternatively, the patient medical history or cardiological data acquired during the patient's visit to the emergency room can cause the patient 12 to be automatically identified as worthy of this on-going assessment, and can therefore cause the patient identification 13 to be flagged accordingly. Once flagged, the SCD risk assessment tool 30 can calculate the probability of SCD p which can be stored in the MUSE system 20, along with any newly-acquired patient data. An appropriate healthcare provider can be alerted and/or consulted if the probability of SCD p represents a risk of SCD that is greater than a pre-determined threshold.

The patient 12 may also have visited an operating room for a variety of procedures 14. The procedures 14 identified as examples in FIG. 1 include knee surgery, implanting a pacemaker or defibrillator, and cardiac bypass graft surgery (CBGS). Before, during, and/or after any one of these procedures 14, cardiological and other patient data can be acquired from the patient 12. If the patient 12 has been identified as worthy of on-going SCD risk assessment, the SCD risk assessment tool 30 can be used to calculate an updated probability of SCD p. The cardiological and other patient data acquired using the patient monitors 16 in the operating room can be stored in the MUSE system 20, along with the calculated probability of SCD p. As mentioned above, the probability of SCD p can depend on the most recent data or information acquired, or it can depend on any portion of the patient's history stored in or accessed by the MUSE system 20.

In some embodiments of the invention, once the patient 12 has been identified as worthy of on-going SCD risk assessment, the SCD risk assessment tool 30 can be displayed as an icon on any patient monitor 16 associated with or connected to the patient 12. The healthcare provider can click on the icon representing the SCD risk assessment tool 30 in order to run the SCD assessment tool 30, calculate the probability of SCD p for the patient 12, and display the results of the SCD risk assessment on the patient monitor 16. The healthcare provider can select the input parameters that the SCD risk assessment tool 30 uses to calculate the probability of SCD p, or the SCD assessment tool 30 can calculate the probability of SCD p using default settings or a predetermined set of input parameters. Similarly, the healthcare provider can select whether the probability of SCD p is dependent upon the most recent data or information acquired for the patient 12 or upon cumulative data stored in or accessed by the MUSE system 20. Also, the SCD risk assessment tool 30 can be preset to calculate the probability of SCD p either based on most recent data or information or cumulative data stored in or accessed by the MUSE system 20. Alternatively, the SCD assessment tool 30 can calculate the probability of SCD p based on a combination of most recent data and cumulative data stored in or accessed by the MUSE system 20. For example, the SCD risk assessment tool 30 can calculate a first probability of SCD p based on the most-recently acquired data, and then depending on the results of the first calculation, decide (e.g., using an if-then statement) whether a follow-up calculation of the probability of SCD p that depends on any of the previously-acquired data is necessary.

The SCD risk assessment tool 30, in some embodiments, can be formed of two or more separate devices (e.g., hardware and/or software) that receive input parameters from the MUSE system 20, calculate the probability of SCD p, store the probability of SCD p in the MUSE system 20, and alert appropriate healthcare personnel if the probability of SCD p represents a risk of SCD greater than a pre-determined threshold. For example, the SCD risk assessment tool 30 can be a module of the MUSE system 20, a software program that interfaces with the MUSE system 20, or a separate hardware device that can receive data from the MUSE system 20. Regardless of how the SCD risk assessment tool 30 is embodied, the SCD risk assessment tool 30 can also be configured to search for other patient identifications within the MUSE system 20 and/or the hospital information system 24 to find other patient profiles that at least partially match the current patient's data or information. The matching patient profiles can be used as an aid in diagnosing the current patient 12 based on the previous experiences and outcomes of other similarly-situated patients.

Again referring to FIG. 1, the patient 12 may also have visited a cardiology clinic for a variety of symptoms, tests, and/or procedures 14. The tests and/or procedures 14 shown in FIG. 1, by way of example only, include a standard or non-standard electrocardiogram (ECG), a transesophageal echocardiogram (TEE), a volunteer cardiac assessment, a treadmill stress test (TST), a treadmill stress test plus an echocardiogram (TST+echo), a nuclear medicine treadmill stress test (NMTST), a dobutamine stress test (DST), and a nuclear medicine intravenous persantine-thallium stress test (NMIVPST). Before, during, and/or after each of the procedures 14 defined in FIG. 1 as being performed or administered in the cardiology clinic, cardiological or non-cardiological data can be acquired from the patient 12. If the patient 12 has been identified as worthy of on-going SCD risk assessment, the SCD risk assessment tool 30 can be used to calculate an updated probability of SCD p before, during, and/or after each of the above-listed procedures 14. Because a large quantity of cardiological data is likely to be acquired from the patient 12 for any of the above tests and/or procedures 14, the calculation of the probability of SCD p each time new data is acquired allows the MUSE system 20, along with the appropriate healthcare providers, to maintain an updated or even real-time calculation of the probability of SCD p.

The patient 12 may also have visited a sleep disorders clinic for a variety of symptoms, tests and/or procedures 14. The symptoms, tests, and/or procedures 14 shown in FIG. 1, by way of example only, include visiting the sleep disorders clinic for the administration of tests and/or procedures related to sleep apnea. Before, during, and/or after any tests or procedures that may be performed or administered in the sleep disorders clinic, cardiological or non-cardiological data can be acquired from the patient 12, stored in the MUSE system 20, and used to calculate the probability of SCD p using the SCD risk assessment tool 30.

The patient 12 may also have visited a cardiac catheterization lab (“cath lab”) for a variety of symptoms, tests, and/or procedures 14. The tests and/or procedures 14 shown in FIG. 1, by way of example only, include cardiac catheterization, percutaneous transluminal coronary angioplasty (PCTA), and stent placement procedures. Before, during, and/or after any tests or procedures that may be performed or administered in the catheterization lab, cardiological or non-cardiological data can be acquired from the patient 12 and used to calculate the probability of SCD p using the SCD risk assessment tool 30. Similarly, the patient 12 may also have visited an electrophysiology lab (“EP lab”) for a variety of symptoms, tests, and/or procedures 14. The tests and/or procedures 14 shown in FIG. 1, by way of example only, include an electrophysiology study and cardiac ablation therapy. Before, during, and/or after any tests or procedures that may be performed or administered in the electrophysiology lab, cardiological or non-cardiological data can be acquired from the patient 12 and used to calculate the probability of SCD p using the SCD risk assessment tool 30.

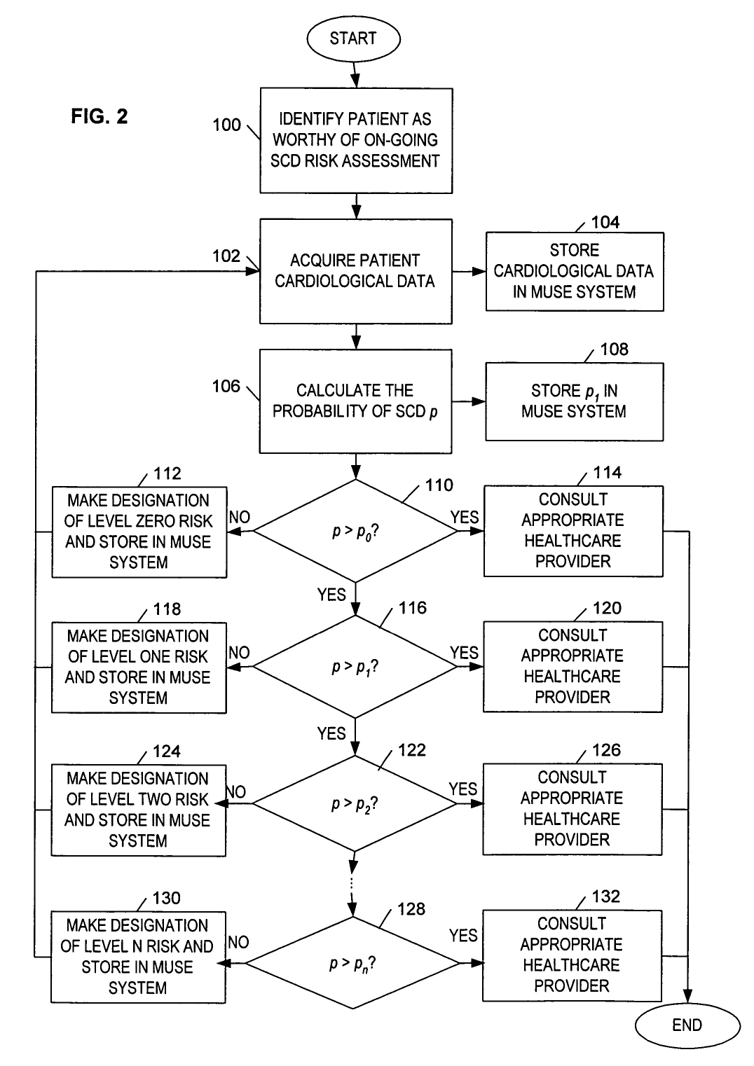

FIG. 2 illustrates a flow chart that represents a SCD assessment method according to one embodiment of the invention. A patient can be identified (at 100) as being worthy of on-going SCD risk assessment. As discussed above, this can be determined manually or automatically such that the patient identification associated with the patient is flagged for on-going SCD risk assessment. Cardiological data can be acquired (at 102) from the patient. The cardiological data acquired from the patient at block 102 can be stored (at 104) in the MUSE system 20. The cardiological data acquired from the patient (at 102) can be acquired during any of the tests and/or procedures 14 and in any of the locations 18 illustrated in FIG. 1 and described above. A SCD risk assessment tool 30, such as that depicted in FIG. 1, can calculate (at 106) the probability of SCD p. The probability of SCD p can be calculated from the cardiological data acquired (at 102), can be calculated based on any of the data that had previously been acquired and stored in the MUSE system 20, or any suitable combination thereof. The probability of SCD p can be stored (at 108) in the MUSE system 20 (for example, with a date and time stamp) to ensure that the MUSE system 20 can be updated with the most-recently calculated probability of SCD p.

The probability of SCD p can be compared (at 110) to a first probability constant of SCD pa that can mark the upper limit of a Level Zero risk of SCD. If the probability of SCD p is less than or equal to the first probability constant pₒ, then the probability of SCD p can be deemed to signify a Level Zero risk of SCD. For example, if pₒ is 0.05 (or 5%), and p is less than pₒ, then p can be deemed a Level Zero risk of SCD. If p is determined (at 110) to be less than or equal to pₒ, the patient's SCD risk can be designated (at 112) as being Level Zero risk, and this designation can also be stored (at 112) in the MUSE system 20. The method can then be repeated (e.g., by returning to block 102) when any additional data or information is acquired from the patient.

However, if p is greater than pₒ, an appropriate healthcare provider can be alerted and/or consulted (at 114). An appropriate healthcare provider can be alerted (at 114) by way of a display on a patient monitor, an audio signal, an e-mail, a phone call, a page, etc. Alerting or consulting the appropriate healthcare provider can include simply alerting a nurse initially that the probability of SCD p is no longer designated as a Level Zero risk and that further analysis may be required. In addition, if p is greater than pₒ, p can be compared (at 116) to a second probability constant p₁, which can mark an upper limit of a Level One risk. If p is less than or equal to p₁, the patient's SCD risk can be designated (at 118) as a Level One risk, and this designation can be stored (at 118) in the MUSE system 20.

However, if p is greater than p₁, an appropriate healthcare provider can be alerted and/or consulted (at 120), as described above. In addition, if p is greater than p₁, p can be compared (at 128) to a third probability constant p₂, which can mark an upper limit of a Level Two risk. If p is less than or equal to p₂, the patient's SCD risk can be designated (at 130) as being a Level Two risk, and this designation can be stored (at 130) in the MUSE system 20. If p is greater than p₂, an appropriate healthcare provider can be alerted and/or consulted (at 132), as described above. This comparison of the probability of SCD p with various probability constants to determine the level of risk of SCD can continue until p is compared (at 128) to the n^{th} probability constant pₙ, which marks the upper limit of a Level N risk.

The calculation (at 106) of the probability of SCD p and the comparisons (at 110, 116, 122, . . . , and 128) of the probability of SCD p to the probability constants can be performed simultaneously with acquiring (at 102) cardiological data from the patient. For example, if a first ECG is acquired from the patient, a second ECG, or other cardiological or non-cardiological data, can be acquired from the patient while the probability of SCD p is being calculated based on the first ECG and initially compared to the probability constants.

The probability constants of SCD (i.e., pₒ, p₁, p₂, . . . , pₙ), can be assigned specific values manually by a healthcare provider, can be assigned default settings in a SCD risk assessment system that is applied to all patients, or can be assigned specific values (i.e., manually or automatically) that pertain to each individual patient. If the specific values of the probability constants are determined automatically for each patient, the number of levels and the magnitude of each level can be determined based on various patient characteristics, such as one or more of the characteristics listed above that the query module 32 uses to determine whether a patient is worthy of on-going SCD risk assessment [e.g., the patient's age, gender, race, family history, weight, blood pressure, a previous cardiac arrhythmia, a low ejection fraction, a pre-existing condition (such as diabetes, mitral valve prolapse, etc.), a previous clinical event (such as an embolism, a heart attack, a stroke, etc.), patient lifestyle (such as stress level, drinking habits, smoking habits, etc.)].

Various healthcare providers can be alerted and/or consulted depending on which level of risk is assigned to the patient. For example, in some embodiments, a Level Zero risk can signify a very low probability such that no healthcare providers need to be alerted and/or consulted. In other embodiments, a Level One risk can still be deemed to signify a relatively low probability of SCD such that no healthcare providers need to be alerted and/or consulted. However, any level of risk greater than a Level One risk may signify that a healthcare provider, such as a cardiologist, should always be alerted and/or consulted.

In still other embodiments, a different healthcare provider can be alerted and/or consulted for each time the probability of SCD p is found to be greater than any one of the probability constants (i.e., pₒ, p₁, p₂, . . . , or pₙ). For example, referring to FIG. 2, a nurse can be alerted and/or consulted (at 114) for lower levels of risk, or a cardiologist can be alerted and/or consulted (at 120, 126, . . . , and 132) for higher levels of risk.

Various features and advantages of the invention are set forth in the following claims.

1. A method of assessing a risk of sudden cardiac death for a patient, the method comprising:
acquiring patient data at one of a plurality of healthcare locations;
identifying the patient as being worthy of an on-going sudden cardiac death risk assessment based on the acquired patient data; and
performing the on-going sudden cardiac death risk assessment whenever new patient data is acquired at any one of the plurality of healthcare locations.

2. The method of claim 1 and further comprising performing the on-going sudden cardiac death risk assessment in real-time whenever new patient data is acquired.

3. The method of claim 1 and further comprising automatically performing the on-going sudden cardiac death risk assessment whenever new patient data is acquired.

4. The method of claim 1 and further comprising acquiring patient data including at least one of cardiological patient data and non-cardiological patient data.

5. The method of claim 1 and further comprising accessing a sudden cardiac death risk assessment tool via an icon displayed on a patient monitor.

6. The method of claim 1 and further comprising acquiring patient data at one of a plurality of healthcare locations including at least one of a patient's home, an emergency room, an operating room, a cardiology clinic, a sleep disorders clinic, a catheterization laboratory, and an electrophysiology laboratory.

7. The method of claim 1 and further comprising displaying an assessment of sudden cardiac death risk on a patient monitor located at one of the plurality of healthcare locations.

8. The method of claim 1 and further comprising storing the acquired patient data in a hospital information system and accessing the acquired patient data from the hospital information system in order to perform the on-going sudden cardiac death risk assessment.

9. The method of claim 1 and further comprising calculating a probability of sudden cardiac death for the patient based on at least one of the new patient data and a medical history of the patient.

10. The method of claim 9 and further comprising alerting a healthcare provider if the probability of sudden cardiac death is greater than a threshold.

11. The method of claim 9 and further comprising comparing the probability of sudden cardiac death to at least one probability constant to determine a risk level.

12. The method of claim 11 and further comprising selecting the at least one probability constant for a specific patient.

13. The method of claim 1 and further comprising performing the on-going sudden cardiac death risk assessment based on at least one of an electrocardiogram, an echocardiogram, cardiac imaging, a stress test, a stress-echocardiogram, a stress-nuclear test, a cardiac catheterization study, an electrophysiology study, and a Holter study.

14. The method of claim 1 and further comprising performing the on-going sudden cardiac death risk assessment based on measurements including at least one of blood pressure, temperature, respiration rate, carbon dioxide, oxygen saturation, and weight.

15. The method of claim 1 and further comprising identifying the patient as being worthy of an on-going sudden cardiac death risk assessment based on at least one of age, gender, race, family history, weight, blood pressure, an arrhythmia, ejection fraction, a pre-existing condition, a previous embolism, and patient lifestyle.

16. The method of claim 1 and further comprising flagging an identification associated with the patient if the patient is worthy of an on-going sudden cardiac death risk assessment.

17. The method of claim 1 and further comprising updating the on-going sudden cardiac death risk assessment whenever new patient data is acquired at any one of the plurality of healthcare locations.

18. The method of claim 1 and further comprising selecting at least one input parameter upon which the on-going sudden cardiac death risk assessment is performed.

19. The method of claim 1 and further comprising identifying a profile of another patient that at least partially matches the new patient data.

20. The method of claim 1 and further comprising performing the on-going sudden cardiac death risk assessment based upon at least one of T-wave alternans, QRS duration, an electrocardiogram serial comparison, an arrhythmia, heartrate turbulence, signal-averaged electrocardiograms, rhythm abnormalities, ST/T measurements, and heart rate variability.

21. A method of identifying a patient for an on-going sudden cardiac death risk assessment, the method comprising:
searching patient data stored in a hospital information system for a characteristic that identifies the patient as being worthy of an on-going sudden cardiac death risk assessment; and
flagging an identification associated with the patient if the characteristic indicates that the patient is at risk for sudden cardiac death.

22. The method of claim 21 and further comprising performing the on-going sudden cardiac death risk assessment whenever new patient data is acquired at any one of a plurality of healthcare locations.

23. The method of claim 21 and further comprising performing the on-going sudden cardiac death risk assessment in real-time whenever new patient data is acquired.

24. The method of claim 21 and further comprising automatically performing the on-going sudden cardiac death risk assessment whenever new patient data is acquired.

25. The method of claim 21 and further comprising acquiring patient data including at least one of cardiological patient data and non-cardiological patient data.

26. The method of claim 21 and further comprising accessing a sudden cardiac death risk assessment tool via an icon displayed on a patient monitor.

27. The method of claim 21 and further comprising acquiring patient data at one of a plurality of healthcare locations including at least one of a patient's home, an emergency room, an operating room, a cardiology clinic, a sleep disorders clinic, a catheterization laboratory, and an electrophysiology laboratory.

28. The method of claim 21 and further comprising displaying an assessment of sudden cardiac death risk on a patient monitor located at one of a plurality of healthcare locations.

29. The method of claim 21 and further comprising accessing the acquired patient data from the hospital information system in order to perform the on-going sudden cardiac death risk assessment.

30. The method of claim 21 and further comprising calculating a probability of sudden cardiac death for the patient based on at least one of new patient data and a medical history of the patient.

31. The method of claim 30 and further comprising alerting a healthcare provider if the probability of sudden cardiac death is greater than a threshold.

32. The method of claim 30 and further comprising comparing the probability of sudden cardiac death to at least one probability constant to determine a risk level.

33. The method of claim 32 and further comprising selecting the at least one probability constant for a specific patient.

34. The method of claim 21 and further comprising performing the on-going sudden cardiac death risk assessment based on at least one of an electrocardiogram, an echocardiogram, cardiac imaging, a stress test, a stress-echocardiogram, a stress-nuclear test, a cardiac catheterization study, an electrophysiology study, and a Holter study.

35. The method of claim 21 and further comprising performing the on-going sudden cardiac death risk assessment based on measurements including at least one of blood pressure, temperature, respiration rate, carbon dioxide, oxygen saturation, and weight.

36. The method of claim 21 and further comprising identifying the patient as being worthy of an on-going sudden cardiac death risk assessment based on at least one of age, gender, race, family history, weight, blood pressure, an arrhythmia, ejection fraction, a pre-existing condition, a previous embolism, and patient lifestyle.

37. The method of claim 21 and further comprising updating the on-going sudden cardiac death risk assessment whenever new patient data is acquired at any one of a plurality of healthcare locations.

38. The method of claim 21 and further comprising selecting at least one input parameter upon which the on-going sudden cardiac death risk assessment is performed.

39. The method of claim 21 and further comprising identifying a profile of another patient that at least partially matches data for the patient.

40. The method of claim 21 and further comprising performing the on-going sudden cardiac death risk assessment based upon at least one of T-wave alternans, QRS duration, an electrocardiogram serial comparison, an arrhythmia, heartrate turbulence, signal-averaged electrocardiograms, rhythm abnormalities, ST/T measurements, and heart rate variability.
